(19) United States
(12) Patent Application Publication
(10) Pub. No.: US 2025/0268935 A1
(43) Pub. Date: Aug. 28, 2025

(54) Tumor Treatment

(71) Applicant: Harold E. Siess, Ooltewah, TN (US)

(72) Inventor: Harold E. Siess, Ooltewah, TN (US)

(21) Appl. No.: 19/207,849

(22) Filed: May 14, 2025

Related U.S. Application Data
(63) Continuation-in-part of application No. 17/249,734, filed on Mar. 11, 2021.
(60) Provisional application No. 62/987,912, filed on Mar. 11, 2020.

(51) Int. Cl. A61K 33/38; A61K 31/375; A61K 31/5415; A61K 31/675; A61K 33/04; A61K 36/87

(52) U.S. Cl. CPC A61K 33/38; A61K 31/375; A61K 31/5415; A61K 31/675; A61K 33/04; A61K 36/87

(57) ABSTRACT

A method for treating a tumor in a host in vivo, where the tumor has a centralized necrotic region, an intermediate hypoxic region, and a peripheral vascularized region. An antidotal agent is administered to the host intravenously, and a period of time is allowed for the antidotal agent to perfuse into vascularized cells in the host. A cell-killing agent is administered directly into the centralized region of the tumor, whereby hydrostatic pressure within the tumor forces the cell-killing agent out of the centralized region of the tumor and through the intermediate region of the tumor in an interstitial flow, killing any non-vascularized cells in the centralized region and the intermediate region. The cell-killing agent is neutralized by the antidotal agent as the cell-killing agent passes through the vascularized cells in the peripheral region.

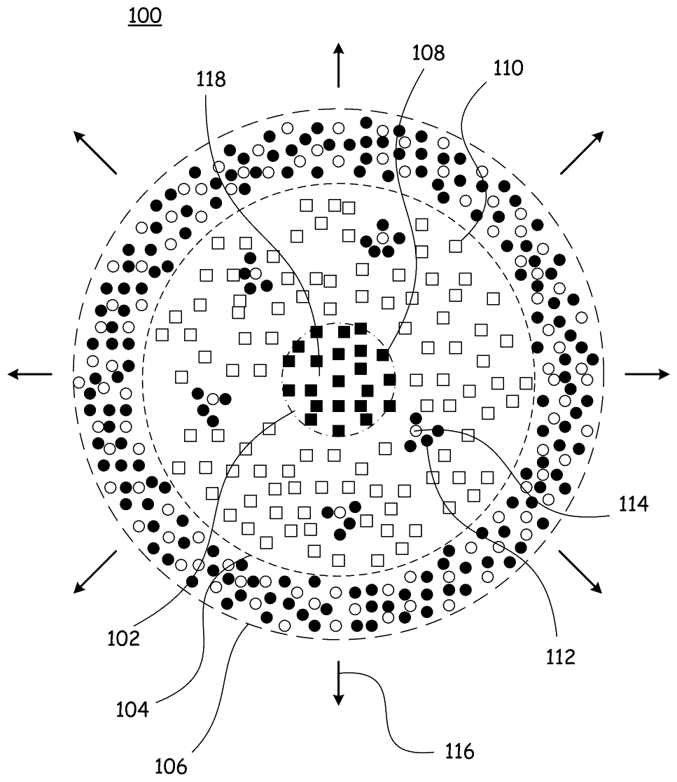

### PRIORITY CLAIM

This application is a continuation-in-part of, and claims priority on prior pending U.S. non-provisional patent application Ser. No. 17/249,734 filed 2021 Mar. 11, which further claims priority on U.S. provisional patent application Ser. No. 62/987,912 filed 2020 Mar. 11, the entirety of the disclosures of which are incorporated herein by reference.

### FIELD

This invention relates to the field of the treatment of tumors. More particularly, this invention relates to a method of treating individual tumors with a cell-killing agent that is neutralized upon exiting the tumor.

### INTRODUCTION

Proliferative-cell tumors such as cancers are difficult to treat for a variety of reasons. One reason is that it can be difficult to get the treating agent into the tumor. Another reason is that the treating agent is typically toxic not only to the cancer cells, but also to the normal, healthy cells of the host.

What is needed, therefore, is a treatment method that reduces issues such as those introduced above, at least in part.

### SUMMARY

The above and other needs are met by a method for treating a tumor in a host in vivo, where the tumor has a centralized necrotic region, an intermediate hypoxic region, and a peripheral vascularized region. An antidotal agent is administered to the host intravenously, and a period of time is allowed for the antidotal agent to perfuse into vascularized cells in the host. A cell-killing agent is administered directly into the centralized region of the tumor, whereby hydrostatic pressure within the tumor forces the cell-killing agent out of the centralized region of the tumor and through the intermediate region of the tumor in an interstitial flow, killing any non-vascularized cells in the centralized region and the intermediate region. The cell-killing agent is neutralized by the antidotal agent as the cell-killing agent passes through the vascularized cells in the peripheral region.

In some embodiments, the antidotal agent comprises silver. Some embodiments include, prior to the step of administering the antidotal agent, administering methylene blue and a super-physiological dose of vitamin C to the tumor, thereby producing leucomethylene blue and dehydroascorbic acid within the tumor. Some embodiments include administering at least one of grape seed proanthocyanidins and a lipid-soluble antioxidant. In some embodiments, the cell-killing agent includes an injectable selenite-containing hydrogel. In some embodiments, the cell-killing agent includes an H2S donor, with at least one of sodium hydrosulfide, sodium sulfide, and GYY4137.

### BRIEF DESCRIPTION OF THE DRAWING

Further advantages of the invention are apparent by reference to the detailed description when considered in conjunction with the FIGURE, which is not to scale so as to more clearly show the details, and which depicts a tumor.

### DESCRIPTION

With reference now to the drawing, there are depicted all of the claimed elements of the various embodiments. It is appreciated that not all embodiments include all of the elements as depicted, and that some embodiments include different combinations of the depicted elements. It is further appreciated that the various elements can all have many different configurations, and are not limited to just the configuration of a given element as depicted. As indicated above, the elements of the drawing as depicted are not to scale, even with respect one to another, and relative size or thickness of one element cannot be determined by the aspect ratios of that element or with reference to any dimension of another element.

One embodiment of the present disclosure kills cancer tumors using at least one cell-killing agent and at least one antidotal agent that is effective at neutralizing the cell-killing agent.

### Delivery Method

The FIGURE depicts a tumor 100 disposed, for example, in healthy tissue in a human body. In general, the tumor 100 has three basic regions, including a centralized necrotic region 102, an intermediate hypoxic region 104, and a peripheral region 106. Within the necrotic region 102 are disposed mostly necrotic cancer cells 108, indicated by black squares. Within the hypoxic region 104 are disposed mostly hypoxic cancer cells 110, indicated by white squares. However, also disposed within the hypoxic region 104 are a relatively fewer number of blood capillaries 114, indicated by white circles. Because of the greater degree of perfusion, a higher level of oxygen is provided by these capillaries 114 in the hypoxic region 104, a few oxygenated and well perfused cancer cells 112 are disposed, indicated by black circles. Within the peripheral region 106 there are disposed a relatively greater number of blood capillaries 114, and a relatively greater number of well oxygenated and well perfused cancer cells 112.

Because of the presence of the capillaries 114, the comparatively well perfused cancer cells 110 within the hypoxic region 104 are capable of accumulating some amount of any antidotal agent that might be delivered thereby, as explained in more detail hereinafter. The well perfused and oxygenated cancer cells 112 in the peripheral region 106 are similarly able to accumulate any antidotal agent that might be delivered via the capillaries 114.

The tumor 100 exhibits a relatively higher uniform hydrostatic pressure at the inner-more portions of the tumor 100, most especially at the necrotic region 102, and a relatively lower hydrostatic pressure at the outer-more portions of the tumor 100, most especially at the well-vascularized peripheral region 106. This gradient in hydrostatic pressure explains, at least in part, the lack of capillaries 114 in the necrotic region 102, a slightly increased number of capillaries 114 in the hypoxic region 104, and a greater number of capillaries 114 in the peripheral region 106. This also explains, at least in part, the necrosis of the cancer cells 108 in the necrotic region 102, the hypoxia of the cancer cells 110 in the hypoxic region 104, and the oxygenated cancer cells 112 (predominantly) in the peripheral region 106 of the tumor 100.

Further, this also explains, at least in part, the difficulty in killing the tumor 100 via an introduction of a tumor-killing agent through the vascular system (through the capillaries 114). The vascular system is not able to adequately infuse the tumor-killing agent through the necrotic region 104 and into the necrotic cancer cells 108, nor substantially into the hypoxic region 104 and into the hypoxic cancer cells 110 because of the relative dearth of capillaries 114 and a lack of a functional lymphatic system therein.

Because of this pressure gradient, the interstitial fluid flow 116 radiates outward from the hypoxic region 104 of the tumor 100 and out through the peripheral region 106. After passing through the peripheral portion 106, the interstitial fluid 116 flows through normal, healthy cells in which the tumor 100 is disposed, and into the lymphatic system.

As a preceding step, the antidotal agent is administered to a patient with a proliferative disorder, such as cancer, either systemically or via intra-arterial injection. The antidotal agent is taken up by the well-vascularized cancer cells 112, because of their close proximity to the capillaries 114. The so deposited antidotal agent forms, in effect, a wall of sorts within the peripheral region 106 that comprises antidote-doped cancer cells 112, through which the interstitial fluid 116 flows on its way out of the tumor 100.

In a following step, a stream of the conjugate cell-killing agent 202 is injected into the central hypoxic region 102 of the tumor 100. This agent moves with the interstitial flow 116 past the already-dead necrotic cancer cells 108 in the necrotic region 102, and through the hypoxic region 104, thus killing the hypoxic cells 110. As the cell-killing agent 202 passes through the antidote-doped tumor cancer cells 112 of the peripheral region 106, it is detoxified by the antidotal agent. This detoxified fluid then moves through the healthy normal cells of the host and into the lymphatic vessels. It will be understood by those skilled in the art, that a certain level of the antidotal agent will remain within the blood stream and therefore will also help detoxify the stream as it moves past the capillaries. This is because some of the fluid flow from the inner tumor will enter into the capillaries after passing through the well perfused cancer cells.

In this manner, the cell-killing agent 202 substantially kills the hypoxic cancer cells 110 of the tumor 100 that cannot be adequately reached through the vascular system of the host. Further, the cell-killing agent 202 is detoxified before it can do substantial damage to the health, non-cancerous cells of the host that surround the tumor 100 or those types of organs and tissues that are particularly susceptible to the cell-killing agent, typically the kidneys or brain. As a subsequent step in some embodiments, a standard chemo regimen is administered to the host, which kills the oxygenated cancer cells 112 of the tumor 100, and all of the dead cancer cells of the tumor 100 are disposed of by the normal functions of the host or through the assistance of dialysis. Thus, a method is presented for killing a tumor 100 in which the hypoxic cancer cells 110 cannot otherwise be contacted by the vascular system of the host. An additional subsequent step would be to irradiate the tumor with radiation for killing the well-oxygenated cells, this normally does not kill the hypoxic cells.

### Cell-Killing and Antidotal Agent Combinations

In one embodiment, the antidotal agent contains silver, and deposits minute silver containing particles throughout the well perfused portion of the tumor 100, including the peripheral region 106 of the tumor 100, thereby effectively encapsulating the tumor 100 with what can be called an antidotal chemical barrier. In various embodiments, the conjugate cell-killing agent 118 consists of an injectable selenite-containing hydrogel designed to trigger the generation of reactive oxygen species (ROS) and reactive nitrogen species (RNS), disrupt NADPH homeostasis, and radiosensitizer the hypoxic region 104 of the tumor 100.

Many tissues in the human body produce low levels of hydrogen sulfide, which plays a significant role in DNA repair and mitochondrial ATP production. Tumors generate slightly higher levels of hydrogen sulfide, which remain beneficial to cancer cells. Consideration has been given to developing various chemical classes of anticancer hydrogen sulfide (H2S) donors to initiate mitochondrial cell death pathways in cancerous tumor cells.

Hydrogen sulfide exhibits radioprotective properties at low concentrations, primarily due to increased oxygen consumption within cellular mitochondria. However, at higher concentrations, hydrogen sulfide inhibits oxidative phosphorylation, elevating intracellular oxygen levels. Elevated oxygen levels sensitize cells to ionizing radiation by producing superoxide anion radicals.

Thus, the cell-killing agent 118 may be an H2S donor, such as sodium hydrosulfide (NaHS), sodium sulfide (NaS), or GYY4137. H2S donors specifically induce partial G2/M arrest and promote apoptosis.

Protecting the Host from Silver

In one embodiment, methylene blue is administered to accumulate within the tumor, followed by super-physiological doses of vitamin C to produce leucomethylene blue and dehydroascorbic acid within the tumor. Leucomethylene blue acts as a vitamin E mimic, while the dehydroascorbic acid enters into cells and is reduced to ascorbic acid. This process, combined with super-physiological ascorbic acid and vitamin C levels, protects both the kidneys and liver against the toxic effects of silver ions. The rapid reduction of silver ions to their metallic state further reduces toxicity. To further mitigate silver ion toxicity, at least one additional antioxidant—such as grape seed proanthocyanidins, a lipid-soluble antioxidant like vitamin E,—is administered.

In one embodiment, agents are administered to reduce the toxic effects of silver on the liver, including dietary grape seed proanthocyanidins at a dosage of 100 to 500 mg/kg body weight. Proanthocyanidins protect against lipid peroxidation by acting as antioxidants, scavenging free radicals, and influencing signaling pathways like Nrf2 to enhance antioxidant defenses. In some embodiments, the proanthocyanidins are administered several days before, during, and after the treatment process.

Studies demonstrate the protective effects of grape seed extract and proanthocyanidins. Grape seed extract and polydatin reduce malondialdehyde (MDA) levels in blood and liver and increase antioxidant potential in cadmium-treated rats. Grape seed extract mitigates cadmium-induced neurotoxicity in male albino rats. Grape seed proanthocyanidin extract alleviates arsenic-induced oxidative reproductive toxicity in male mice. Proanthocyanidins attenuate chronic lead-induced liver oxidative damage in Kunming mice via the Nrf2/ARE pathway. The hepatoprotective effects of grape seed proanthocyanidins on cadmium-induced hepatic injury in rats has been confirmed, involving mitochondrial dysfunction, inflammation, and apoptosis. Procyanidins are potent antioxidants, surpassing vitamins C and E, by enhancing antioxidative enzyme activity, reducing lipid peroxidation, and increasing total antioxidant capacity (T-AOC) through competitive binding of active oxygen radicals. In addition, to protecting the liver, proanthocyanidins have also shown potential in protecting the kidneys from the damaging effects of heavy metals, particularly cadmium, one of the most toxic heavy metals. A common feature of heavy metal poisoning is the development of lipid peroxidation. Proanthocyanidins, particularly oligomeric proanthocyanidin complexes, have been shown to help prevent lipid peroxidation.

### Other Applications of Silver

This disclosure outlines methods and compositions for treating different conditions. The first set of methods targets unwanted cells, such as cancerous cells, bacteria, virally infected cells, or malaria parasites. For cancer, these methods aim to achieve successful outcomes, particularly for challenging types of cancer such as triple-negative breast cancer, which current treatments struggle to address. The cancer treatment comprises different methods, some of which can be combined for therapeutic effect.

The first method eliminates cancer cells by generating an avalanche of dehydroascorbic acid (DHA) that targets proteins with reductive amino acids, such as antioxidants like glutathione or cysteine containing or other reducing group enzymes critical to cell survival. Previous efforts over the past fifty years have involved administering high-dose ascorbic acid, some of which oxidizes in the tumor's oxidative environment. Current efforts continue to refine these approaches. Embodiments of the present disclosure employ methylene blue to oxidize ascorbic acid within the tumor, creating an avalanche of DHA that overwhelms the reductive capacity of cancer cells, leading to the attack of essential proteins containing one or more reducing groups.

### Unwanted Cells

### Generating an Avalanche of DHA in Cancer Cells

### Step One: Administering Methylene Blue

The method begins by administering methylene blue, an oxidizer, to the patient, forming an oxidative cloud of methylene blue within the tumor's components, including cancerous cells. Methylene blue molecules attach to various tumor structures, such as collagen, both inside and outside cells. Unlike most oxidizers, methylene blue remains non-toxic at low concentrations. Notably, methylene blue triggers the upregulation of GLUT1 transporters, which many cancer cells already overexpress. This upregulation enhances the transport of DHA into cancer cells. For patients with tumors already exhibiting upregulated GLUTs capable of transporting DHA, methylene blue's role in further upregulating GLUT1 becomes less critical but remains beneficial.

At high concentrations, methylene blue can oxidize hemoglobin's iron to the +3 state, impairing oxygen transport. Methylene blue is a cation that can bind with the negative charges upon cell membranes and on negatively charged tissue components, such as collagen. Cancerous tumors have a high degree of negative charge associated with them due to the increased glycolysis and associated lactate production within tumors.

Within the body's cells, including cancer cells, fibroblasts, and macrophages, some methylene blue reduces to leucomethylene blue, a colorless, lipophilic compound. Leucomethylene blue's increased mobility allows it to move freely in and out of cells and exit the body via urine. However, some studies have shown that individuals with ongoing inflammation may excrete methylene blue also in its oxidized form. This reoxidation sustains high methylene blue concentrations in the tumor, even as other normal tissues accumulate and subsequently release it as leucomethylene blue into the bloodstream. The extended diffusional distance between cancer cells and tumor-feeding capillaries further maintains elevated methylene blue and leucomethylene blue levels in the tumor.

### Step Two: Administering Ascorbic Acid

After methylene blue and leucomethylene blue levels in the blood or urine reach a lower plateau, indicating excretion from non-tumor tissues, ascorbic acid is administered to achieve super-physiological blood levels. Super-physiological levels exceed those attainable through oral administration of standard vitamin C. Three administration methods achieve this: (1) intravenous (IV) ascorbic acid, (2) oral nanoparticles of ascorbic acid that bypass intestinal absorption barriers, and (3) inhalation of ascorbic acid. The preferred method involves IV administration. The dose of vitamin C given can be the same as that used in the traditional use of IV administered Vitamin C for the treatment of cancer.

As ascorbic acid enters the tumor, it oxidizes in the extracellular fluid by both the abundance of reactive oxidative species normally present within tumor and by the methylene blue that has accumulated in the tumor into DHA, which enters cancer cells via GLUT transporters, initiating the DHA avalanche. Cancer cells can only reduce so much of the DHA, but it reoxidizes to DHA, sustaining the avalanche within the cells to cytotoxic levels.

### Using Silver in Cancer Treatment

The next three methods employ metallic species, primarily silver compounds, leveraging the shared transport mechanisms for silver and copper ions. In aqueous environments, copper typically exists as Cu²⁺ ions, but within bacteria and human cells, including cancer cells, copper is handled as Cut. The mechanisms enabling silver ions to use copper transporters are detailed later in this application.

This second method selects patients with cancerous conditions or proliferative disorders exhibiting upregulated copper transporters for silver-based treatment. Methylene blue and high-dose ascorbic acid are administered to achieve three objectives. First, methylene blue upregulates glucose transporters in tumor cells to the extent possible. Second, methylene blue oxidizes ascorbic acid in the serum, forming DHA that tumor cells absorb, creating an intracellular ascorbic acid pool. Preferably, ascorbic acid is administered in excess of methylene blue, forming a protective ascorbic acid cloud in normal cells to shield them from the toxic effects of subsequently administered silver. Third, ascorbic acid reduces methylene blue to leucomethylene blue, which diffuses into both normal and tumor cells, forming a protective barrier against lipid peroxidation caused by silver administration.

After administering methylene blue and ascorbic acid, a silver compound is introduced to deposit silver within the tumor's unwanted cells environment, including cancer cells. This deposited silver subsequently undergoes dissolution, forming a cloud of silver ions within the tumor. In one embodiment, the silver compound reacts with the intracellular ascorbic acid and leucomethylene blue to form a silver deposit. Preferably, the silver is administered as a salt, such as a methylene-silver salt, which dissociates in the bloodstream into methylene blue and silver ions. This form is less toxic than silver nitrate, as the methylene blue reduces to leucomethylene blue within the body. The rate of dissolution of silver from the deposits formed within in the tumor can be controlled by adjusting the patient's oxygen partial pressure, with higher oxygen levels accelerating dissolution.

### Creating Chemical Barriers for Targeted Cancer Treatment

This method administers a first substance to form chemical barriers in specific tissues, limiting the regional toxicity of a therapeutic agent to a single tissue type. The first substance may chemically combine with the therapeutic agent to form a less toxic product or create a complex, such as through chelation.

The method establishes a chemical barrier by depositing silver in the fast-growing, well-perfused outer regions of a cancerous tumor using the techniques described above. This barrier restricts the therapeutic agent's toxic effects to the targeted tumor tissue.

### Controlling Copper Release for Cuproptosis in Cancer Treatment

Cuproptosis, a distinct cell death mechanism from apoptosis, involves copper binding to lipoylated proteins in the tricarboxylic acid (TCA) cycle, causing protein aggregation and cell death. While copper effectively kills cancer cells, its systemic toxicity poses challenges. Current copper nanoparticle approaches lack control over copper ion release rates, limiting their efficacy. This method uses silver ions to regulate copper ion release from copper nanoparticles via ion exchange, addressing this limitation.

Silver induces cell death through lipid peroxidation, distinct from cuproptosis. The degree of lipid peroxidation can be mitigated, and normal cells can be protected from silver's toxic effects while allowing cuproptosis to occur in tumor cells. Insights from cisplatin, a cancer drug with dual mechanisms (DNA damage and oxidative stress-induced lipid peroxidation), inform this approach. Co-administering high-dose vitamin C with cisplatin reduces side effects by mitigating lipid peroxidation in normal cells while preserving tumor cell death via DNA damage. This disclosure describes a method involving two toxic agents—copper and silver—where silver energizes copper's release, and a third agent (e.g., ascorbic acid) mitigates silver's toxicity without interfering with copper-induced cuproptosis. At high concentrations, ascorbic acid can be detrimental to cancer cells, including those undergoing cuproptosis. Conversely, at lower concentrations, it can act as an antioxidant, potentially interfering with cuproptosis. In the preferred form of the invention supraphysiological levels of ascorbic acid are administered for both protecting normal cells from the toxic effects of silver and for aiding in the process of cuproptosis.

The method administers copper nanoparticles with targeting modalities to concentrate them in tumors. Silver ions are then introduced systemically to release copper ions from the nanoparticles via ion exchange, controlling the release rate. To mitigate silver's toxicity, high-dose ascorbic acid is co-administered, forming a protective barrier against lipid peroxidation in normal cells. This allows cuproptosis to selectively kill tumor cells. The method claims a broad approach: (a) administering two toxic substances, with one acting as an energizing agent for the other, and (b) co-administering a third agent to reduce the energizing agent's toxicity without affecting the primary mode of cell death.

### Silver for Chemical and Biological Agent Exposure

This method treats individuals exposed to chemical or biological agents, such as botulism toxin or sarin nerve gas, using silver-based compositions. Historically, the U.S. Navy used silver prophylactically to prevent syphilis in sailors, achieving significant success. This application adapts these principles with modern techniques for improved efficacy. In one embodiment, dehydroascorbic acid and leucomethylene blue is formed in the body for the treatment of COVID-19. In another embodiment, silver is administered for reacting with the reducing groups in TNF-alpha and IL6. As far as COVID-19 is concerned, silver degrades both TNF-alpha and IL6 by reacting with the cysteine residue at the active site of these messenger molecules.

As used herein, the phrase “at least one of A, B, and C” means all possible combinations of none or multiple instances of each of A, B, and C, but at least one A, or one B, or one C. For example, and without limitation: Ax1, Ax2+Bx1, Cx2, Ax1+Bx1+Cx1, Ax7+Bx12+Cx113. It does not mean Ax0+Bx0+Cx0.

The foregoing description of embodiments for this invention has been presented for purposes of illustration and description. It is not intended to be exhaustive or to limit the invention to the precise form disclosed. Obvious modifications or variations are possible in light of the above teachings. The embodiments are chosen and described in an effort to provide illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A method for treating a tumor in a host in vivo, where the tumor has a centralized necrotic region, an intermediate hypoxic region, and a peripheral vascularized region, the method comprising the steps of:
administering an antidotal agent to the host intravenously,
waiting for a period of time for the antidotal agent to perfuse into vascularized cells in the host, and
administering a cell-killing agent directly into the centralized region of the tumor,
whereby hydrostatic pressure within the tumor forces the cell-killing agent out of the centralized region of the tumor and through the intermediate region of the tumor in an interstitial flow, killing any non-vascularized cells in the centralized region and the intermediate region, and
whereby the cell-killing agent is neutralized by the antidotal agent as the cell-killing agent passes through the vascularized cells in the peripheral region.

2. The method of claim 1, wherein the antidotal agent comprises silver.

3. The method of claim 2, further comprising, prior to the step of administering the antidotal agent:
administering methylene blue to the tumor, and
administering a super-physiological dose of vitamin C to the tumor, thereby producing leucomethylene blue and dehydroascorbic acid within the tumor.

4. The method of claim 3, further comprising administering at least one of grape seed proanthocyanidins and a lipid-soluble antioxidant.

5. The method of claim 1, wherein the cell-killing agent comprises an injectable selenite-containing hydrogel.

6. The method of claim 1, wherein the cell-killing agent comprises an H2S donor, including at least one of sodium hydrosulfide, sodium sulfide, and GYY4137.

7. A method for treating a tumor in a host in vivo, where the tumor has a centralized necrotic region, an intermediate hypoxic region, and a peripheral vascularized region, the method comprising the steps of:
administering methylene blue to the tumor,
administering a super-physiological dose of vitamin C to the tumor, thereby producing leucomethylene blue and dehydroascorbic acid within the tumor,
administering a silver-containing antidotal agent to the host intravenously,
waiting for a period of time for the antidotal agent to perfuse into vascularized cells in the host, and
administering a cell-killing agent directly into the centralized region of the tumor,
whereby hydrostatic pressure within the tumor forces the cell-killing agent out of the centralized region of the tumor and through the intermediate region of the tumor in an interstitial flow, killing any non-vascularized cells in the centralized region and the intermediate region, and
whereby the cell-killing agent is neutralized by the antidotal agent as the cell-killing agent passes through the vascularized cells in the peripheral region.

8. The method of claim 7, further comprising administering at least one of grape seed proanthocyanidins and a lipid-soluble antioxidant.

9. The method of claim 7, wherein the cell-killing agent comprises an injectable selenite-containing hydrogel.

10. The method of claim 7, wherein the cell-killing agent comprises an H2S donor, including at least one of sodium hydrosulfide, sodium sulfide, and GYY4137.

11. A method for treating a tumor in a host in vivo, where the tumor has a centralized necrotic region, an intermediate hypoxic region, and a peripheral vascularized region, the method comprising the steps of:
administering methylene blue to the tumor,
administering a super-physiological dose of vitamin C to the tumor, thereby producing leucomethylene blue and dehydroascorbic acid within the tumor,
administering a silver-containing antidotal agent to the host intravenously,
waiting for a period of time for the antidotal agent to perfuse into vascularized cells in the host, and
administering a cell-killing agent directly into the centralized region of the tumor, wherein the cell-killing agent comprises at least one of an injectable selenite-containing hydrogel, an H2S donor, including at least one of sodium hydrosulfide, sodium sulfide, and GYY4137,
whereby hydrostatic pressure within the tumor forces the cell-killing agent out of the centralized region of the tumor and through the intermediate region of the tumor in an interstitial flow, killing any non-vascularized cells in the centralized region and the intermediate region, and
whereby the cell-killing agent is neutralized by the antidotal agent as the cell-killing agent passes through the vascularized cells in the peripheral region.
